(19) United States
(12) Patent Application Publication
(10) Pub. No.: US 2020/0345608 A1
(43) Pub. Date: Nov. 5, 2020

(54) PERSONAL CARE PRODUCT

(71) Applicant: CARGILL, INCORPORATED, Wayzata, MN (US)

(72) Inventors: Sylvie BOUZELOC, Montigny-le-Tilleul (BE); Katarzyna GALCZYNSKA, Mechelen (BE); Arnulf SCHOEPPE, Hamburg (DE)

(73) Assignee: CARGILL, INCORPORATED, Wayzata, MN (US)

(21) Appl. No.: 16/758,545

(22) PCT Filed: Oct. 23, 2018
(86) PCT No.: PCT/US2018/057012

§ 371 Date: Apr. 23, 2020

(30) Foreign Application Priority Data
Oct. 27, 2017 (EP) 17198786.0

(51) Int. Cl. A61K 8/55; A61K 8/06; A61K 8/73; A61K 8/34; A61Q 5/02; A61Q 19/00

(52) U.S. Cl. CPC A61K 8/553; A61K 8/06; A61K 8/73; A61K 2800/43; A61Q 5/02; A61Q 19/001; A61K 2800/524; A61K 8/345

(57) ABSTRACT

A personal care formulation comprising an emulsifier, a vegetable oil and optionally a texturizer, wherein the emulsifier contains an Acetone Insoluble (AI) component containing at least Phosphatidyl Choline (PC) and Phosphatidyl Ethanolamine (PE), wherein the emulsifier has a weight ratio R of at least 5.0.

### CROSS-REFERENCE TO RELATED APPLICATIONS

This application claims the benefit of European Provisional Patent Application No. 17198786.0, filed Oct. 27, 2017 entitled PERSONAL CARE PRODUCT, which are hereby incorporated by reference in their entirety.

### FIELD OF THE INVENTION

The invention relates to a personal care formulation comprising an emulsifier composition, a vegetable oil and a texturizer. The invention further relates to a method of preparing said formulation and to personal care products containing thereof.

### BACKGROUND OF THE INVENTION

Personal care formulations and products containing thereof, include within the context of the present invention, formulations and products in the field of cosmetics or beautification, personal hygiene and care.

The use of synthetic compounds including petroleum-based substances to manufacture conventional personal care formulations brings growing concerns. These compounds are typically harsh substances, like petroleum, aluminum, and lead, all of which require extensive mining. Much of this is done in some of the world's most beautiful and sensitive areas, like the Amazon rainforest, and miles of land are destroyed and stripped of vital wildlife every year.

In this context, the market of natural products for personal care is growing and evolving constantly. With people striving to live a healthier life, personal care products using natural ingredients gain more and more ground. The “natural personal care” industry, favours natural resources, valorises the use of carefully chosen agricultural ingredients and guarantees the respect for the environment.

Many companies strive to develop natural formulations based on natural ingredients, having the same sensorial characteristics and efficiency as conventional formulations. However, while conventional formulations offer a vast range of viscosities and textures and enable the manufacture of products with textures ranging from liquid to solid, natural formulations are much impaired. Finished products containing natural formulations are mostly functionally inferior to mainstream products. There is thus a real trade-off between natural products and their sensorial

properties and appearance. Moreover, this trade-off is often enhanced by a much higher cost for an often inferior product.

JP 2005/290308 discloses a pharmaceutical, food or personal care product comprising an emulsifier composition of an animal original, i.e. extracted from egg yolk using alcohol. U.S. Pat. No. 4,280,996 discloses an intravenous injection containing a vegetable (soybean) oil, water and egg yolk phospholipids. EP 0 885 896 also discloses a phospholipid composition manufactured from dried egg yolk that can be used to emulsify pharmaceutical, cosmetic and food products. Although having excellent properties, the products manufactured using egg-yolk based emulsifier compositions are starting to become less appealing to the consumers. In the recent years, there is a growing desire to having vegan, kosher and halal personal care products with companies holding themselves to higher standards by producing such products which are free from cruelty and animal by-products. Often, such companies also use eco-friendly packaging along with new business models influenced by social enterprise movements. Thus new non-animal based cosmetic ingredients are needed to support this new omnicultural beauty movement process. In addition, such non-animal cosmetic ingredients may be beneficial for those consumers having sensitive skin and religious preferences.

WO 2014/165484 discloses a cosmetic composition comprising an emulsifier composition having a vegetable origin. The present inventors however observed that the properties of said composition can be further optimized, at least in terms of stability and/or appearance. In particular, the inventors could find no vegetable-origin emulsifier composition able to produce an emulsion having the stability and fine particle sizes which can be produced by using an animal-origin emulsifier composition.

In spite of all these technical drawbacks, the demand for natural, vegetable-based products continue to grow and companies try to bring improvements in performance and a drop in cost. There is thus a long-felt need in the field of cosmetic and personal care and hygiene products for natural, vegetable-based formulations which have a reduced toxicity or are devoid of it and enable the manufacture of end products having the right properties, e.g. texture, feel, appearance and stability.

### SUMMARY OF THE INVENTION

The present invention is based on a discovery that certain combinations of natural ingredients of vegetable origin, in certain proportions can lead to formulations having a number of advantageous properties alone or in combination: stability in manufacture, formulation, storage and use; a viscosity optimal for the end use; and preferably a desirable body and good skin feel. Body and skin feel are usually assessed subjectively, and although good body and/or skin feel are commonly associated with a non-Newtonian, shear thinning viscosity profile, a shear thinning profile does not guarantee a good body or skin feel.

The present invention provides a personal care formulation comprising an emulsifier composition, a vegetable oil and optionally a texturizer, wherein the emulsifier composition contains an Acetone Insoluble (AI) component, said AI component containing at least Phosphatidyl Choline (PC) and Phosphatidyl Ethanolamine (PE), wherein the emulsifier composition is of a vegetable origin, wherein the emulsifier composition is characterized by a weight ratio R of at least 5.0, the ratio R being defined according to Formula 1:

$$R = \frac{{W}_{PC}}{{W}_{PE}} \qquad Formula 1$$

wherein W_{PC} and W_{PE} are the individual weights of the respective PC and PE relative to the total weight of the emulsifier composition.

The present inventors noticed that the personal care formulation of the invention (hereinafter referred to for simplicity as the “inventive formulation”) enables the manufacturing of personal care products with textures ranging from liquid, e.g. lotions, syrups, suspensions, solutions, drops, and the like; to semi-solid, e.g. creams, butters, ointments, gels, suppositories and the like; to solid, e.g. powders, bars, and the like. In addition to the ability of the inventive formulation to provide an end product with a desired texture, said formulation also provides to said product optimum sensory properties. Such combination of texture and sensory properties, may enable a designer of end products with the right tools to accurately adjust and balance the product's look, feel and performance.

### DETAILED DESCRIPTION

The present invention provides a personal care formulation comprising an emulsifier composition, a vegetable oil and a texturizer.

For the sake of clarity, it should be noted that the term “formulation” is used herein to denote compositions of various ingredients in various weight ranges, in accordance with the present invention. The formulations are suitable for use on hair, nails and skin, for delivering cosmetic or actives to the skin for providing cleansing, moisturizing, minimizing or treating skin imperfections, reducing skin oiliness, providing fragrances to the hair or skin and the like.

“Personal care” means and comprises any cosmetic, hygienic, toiletry and topical care products including, without limitation, leave-on products (i.e., products that are left on keratinous substrates after application); rinse-off products (i.e., products that are washed or rinsed from keratinous substrates during or within a few minutes of application); shampoos; hair curling and hair straightening products; hair style maintaining and hair conditioning products; lotions and creams for nails, hands, feet, face, scalp and/or body; hair dye; face and body makeup; nail care products; astringents; deodorants; antiperspirants; antiacne; antiaging; depilatories; colognes and perfumes; skin protective creams and lotions (such as sunscreens); skin and body cleansers; skin conditioners; skin toners; skin firming compositions; skin tanning and lightening compositions; liquid soaps; bar soaps; bath products; shaving products; and oral hygiene products (such as toothpastes, oral suspensions, and mouth care products).

The emulsifier composition utilized in accordance with the invention is of a vegetable origin, meaning that the constituents of said composition are produced or extracted from a vegetable source. Any vegetable source, including vegetables, fruits and algae, which can be utilized to extract the necessary phospholipids is suitable for providing the components used in accordance with the invention. Examples of vegetable sources include coconut, corn, cottonseed, canola (rapeseed), olive, palm, peanut (ground nut), safflower, sesame, soybean, sunflower, almond, cashew, hazelnut, macadamia, pecan, pistachio, walnut, acai, blackcurrant, borage seeds, evening primrose, carob seeds, amaranth, apricot, argan, avocado, babassu, ben, carob pod (algaroba), coriander seeds, false flax (made of the seeds of camelina sativa), coriander seeds, hemp, kapok seeds, meadowfoam seeds, mustard, okra seeds, perilla seeds, pine seeds, poppyseeds, prune kernel, pumpkin seeds, quinoa, ramtil, rice bran, tea (camellia), thistle, wheat germs, castor, radish, ramtil, citrus, allanblackia, tung, shea butter, cocoa butter and combinations thereof. Most preferably, the emulsifier composition is extracted from sunflower, soy, rapeseed or combinations thereof. By “extracted from” is herein understood also to include “manufactured from”.

The texture of such personal care formulations is not limited and may be, without limitation, a liquid, gel, spray, emulsion (such as lotions and creams), shampoo, pomade, foam, tablet, stick (such as lip care products), makeup, suppositories, among others, any of which can be applied to the skin or hale and which typically are designed to remain in contact therewith until removed, such as by rinsing with water or washing with shampoo or soap. Other forms could be gels that can be soft, stiff, or squeezable. Emulsions can be oil-in-water, water-in-oil or multiple. Sprays can be non-pressurized aerosols delivered from manually pumped finger-actuated sprayers or can be pressurized aerosols such as mousse, spray, or foam forming formulation, where a chemical or gaseous propellant is used. “Cosmetic” means and comprises a personal care formulation comprising a powder, lotion, lipstick, rouge, or other preparation for affecting the appearance of or beautifying the face, skin, hair, nails or other keratinous materials.

The inventive formulation comprises an emulsifier composition containing Acetone Insoluble (AI) component containing a Phosphatidyl Choline (PC) and a Phosphatidyl Ethanolamine (PE). Preferably, said AI contains in addition to the PC and the PE, a Phosphatidic Acid (PA) and a Phosphatidyl Inositol (PI). The AI component is understood as the component obtained upon mixing the emulsifier composition with acetone, decanting and extracting the acetone in accordance with the method presented herein below in the “Methods for Measurement” section. Preferably the AI component is in a weight ratio of at least 30 wt % relative to the total weight of the emulsifier composition, more preferably at least 45 wt %, most preferably at least 50 wt %. Preferably, the AI is at most 90 wt %, more preferably at most 80 wt %, more preferably at most 70 wt %, more preferably at most 65 wt %, most preferably at most 60 wt %. Preferably, the AI component is between 30 and 90 wt %, more preferably between 40 and 85 wt %, most preferably between 45 and 80 wt %. In one embodiment, AI is preferably between 30 and 70 wt %, more preferably between 45 and 65 wt %, most preferably between 50 and 60 wt % of the total weight of the emulsifier composition.

As used herein, PC, PA, PE and PI are constituents of the emulsifier composition, which are insoluble in acetone and may be commonly referred to as phospholipid fractions or simply as phospholipids. It is herein understood that the individual phospholipid fractions may contain modified and non-modified fractions. Examples of modified phospholipid fraction include hydrogenated PC, PA, PE and PI; and hydrolyzed PC, PA, PE and PI, i.e. lyso-derivatives thereof, hereinafter denoted as L-PC, L-PA, L-PE and L-PI, respectively.

If hydrolyzed phospholipid fractions are present in the emulsifier composition used in accordance with the invention, preferably, said hydrolyzed fractions (i.e. lyso-derivatives of said phospholipid fractions) are present in a total amount of at most 20 wt %, more preferably at most 15 wt %, most preferably at most 8 wt % relative to their non-modified counterparts.

Preferably, in addition to the AI component the emulsifier composition used in accordance with the invention, comprises a lipid component. Preferably, the lipid component is of the same vegetable origin as the phospholipids.

Most preferably, the emulsifier composition used in accordance with the invention contains hydrolyzed PC (L-PC) in an amount of at most 5000 ppm, more preferably at most 4500 ppm, even more preferably at most 4000 ppm, even more preferably at most 3500 ppm, most preferably at most 3000 ppm relative to the total mass of said emulsifier composition. Preferably, said amount is at least 50 ppm, more preferably at least 100 ppm, most preferably at least 150 ppm.

The emulsifier composition used in accordance with the invention has a weight ratio R of at least 5, the ratio R being defined according to Formula 1:

$$R = \frac{{W}_{PC}}{{W}_{PE}} \qquad Formula 1$$

Preferably, R is at least 6, more preferably at least 7, even more preferably at least 8, most preferably at least 9. For practical reasons, R is preferably at most 900, more preferably at most 800, most preferably at most 700. However, advantageous results were obtained when R is at most 100, preferably at most 60, more preferably at most 20, mot preferably at most 18. Preferably, R is between 5 and 100, more preferably between 7 and 60, even more preferably between 7 and 20, most preferably between 7 and 18. In one embodiment, R is between 9 and 20.

The emulsifier composition used in accordance with the invention contains PC in an amount of preferably at least 20 wt % relative to the total weight of said emulsifier composition, more preferably at least 25 wt %, most preferably at least 30 wt %. Preferably said amount of PC is at most 65 wt %, more preferably at most 60 wt %, even more preferably at most 55 wt %, even more preferably at most 50 wt %, even more preferably at most 45 wt %, yet even more preferably at most 40 wt %, most preferably at most 35 wt % relative to the total weight of the emulsifier composition. Preferably the amount of PC is between 20 and 65%, more preferably between 20 and 55 wt %, even more preferably between 25 and 45 wt %. In one embodiment, PC is between 30 and 40 wt %. Preferably, said PC contains, relative to its amount, at most 10.0 wt % of L-PC, more preferably at most 9.0 wt %, even more preferably at most 8.0 wt %, most preferably at most 7 wt %. Preferably, the amount of L-PC is at least 1 wt %, more preferably at least 3 wt %, most preferably at least 5 wt % relative to the total amount of PC.

Preferably, the emulsifier composition has an R of between 5 and 100, an AI between 30 and 90 wt % and a PC content of between 20 and 65 wt %. Preferred ranges for R, AI and PC are given hereinabove and will not be repeated herein.

Preferably the PE in the emulsifier composition is in an amount of at most 10.0 wt %, more preferably at most 8.0 wt %, even more preferably at most 6.0 wt %, most preferably at most 4.0 wt % relative to the total weight of the emulsifier composition. Preferably, said amount of PE is at least 0.1 wt %, more preferably at least 0.5 wt %, even more preferably at least 1.0 wt %, even more preferably at least 1.5 wt %, most preferably at least 2.0 wt %. Preferably the amount of PE is between 0.1 and 10 wt %, more preferably between 0.5 and 6.0 wt %, most preferably between 1.0 and 4.0 wt %.

Preferably, the emulsifier composition has an R of between 5 and 100, an AI between 30 and 90 wt %, a PC content of between 30 and 70 wt % and a PE content of between 0.1 and 10 wt %. Preferred ranges for R, AI, PC and PE are given hereinabove and will not be repeated herein.

Preferably the PA in the emulsifier composition is in an amount of at most 3.0 wt %, more preferably at most 2.5 wt %, even more preferably at most 2.0 wt %, yet even more preferably at most 1.5 wt %, yet even more preferably at most 1.0 wt %, most preferably at most 0.7 wt % relative to the total weight of the emulsifier composition. Preferably, said amount of PA is at least 0.1 wt %, more preferably at least 0.3 wt %, most preferably at least 0.5 wt %.

Preferably, the emulsifier composition has an R of between 5 and 100, an AI between 30 and 90 wt %, a PC content of between 20 and 65 wt %, a PE content of between 0.1 and 10 wt % and a PA content of between 0.1 and 3.0 wt %. Preferred ranges for R, AI, PC, PE and PA are given hereinabove and will not be repeated herein.

Preferably the PI in the emulsifier composition is in an amount of at most 10.0 wt %, more preferably at most 5.0 wt %, most preferably at most 1.0 wt %, relative to the total weight of the emulsifier composition. Preferably, said amount of PI is at least 0.1 wt %, more preferably at least 0.3 wt %, most preferably at least 0.5 wt %.

Preferably, the emulsifier composition has an R of between 5 and 100, an AI between 30 and 90 wt %, a PC content of between 20 and 65 wt %, a PE content of between 0.1 and 10 wt %, a PA content of between 0.1 and 3.0 wt % and a PI content of between 0.1 and 10.0 wt %. Preferred ranges for R, AI, PC, PE, PA and PI are given hereinabove and will not be repeated herein.

Excellent results may be obtained when the emulsifier composition used in accordance with the invention has a total amount of PE, PA and PI of at most 13 wt % relative to the total weight of said emulsifier composition, preferably of at most 10 wt %, more preferably of at most 7 wt %, most preferably of at most 5 wt %.—Preferably said total amount is at least 1 wt %, more preferably at least 2 wt %, most preferably at least 3 wt %. Preferably said total amount is between 1 and 13 wt %, more preferably between 2 and 10 wt %, most preferably between 3 and 7 wt %.

The emulsifier composition used in accordance with the invention is preferably characterized by a weight ratio R′ of at least 4.0, more preferably at least 5.0, most preferably at least 6.0, wherein the ratio R′ is defined according to Formula 2:

$${R}^{′} = \frac{{W}_{PC}}{{W}_{PE + PA + PI}} \qquad Formula 2$$

wherein W_{PC} is the individual weight of PC relative to the total amount of the emulsifier composition and W_{PE+PA+PI} is the sum of the individual weights of the respective phospholipids relative to the total amount of the emulsifier composition. Preferably R′ is at most 100.0, more preferably at most 60.0, most preferably at most 20.0. Preferably, R is between 4.0 and 100.0, more preferably between 5.0 and 60.0, most preferably between 6.0 and 20.0.

Preferably, the emulsifier composition has an R of between 5 and 100, an R′ of between 4.0 and 100.0, an AI between 30 and 90 wt %, a PC content of between 20 and 65 wt %, a PE content of between 0.1 and 10 wt %, a PA content of between 0.1 and 3.0 wt % and a PI content of between 0.1 and 10.0 wt %. Preferred ranges for R, R′, AI, PC, PE, PA and PI are given hereinabove and will not be repeated herein.

The inventive formulation also comprises a vegetable oil. Preferably, the vegetable oil is present in said formulation in an amount of at least 0.1 wt %, more preferably at least 0.5 wt %, even more preferably at least 1.0 wt %, most preferably at least 1.5 wt % relative to the total weight of said formulation. Preferably, the amount of vegetable oil is at most 80.0 wt %, more preferably at most 50.0 wt %, even more preferably at most 30.0 wt %, even more preferably at most 25.0 wt %, most preferably at most 20.0 wt %. Preferably the amount of vegetable oil is between 0.5 and 50.0 wt %, more preferably between 1.0 and 30.0 wt %, most preferably between 1.5 and 20.0 wt %.

Any vegetable oil can be used in the inventive formulation, non-limiting examples thereof including coconut oil, corn oil, cottonseed oil, canola oil (rapeseed oil), olive oil, palm oil, peanut oil (ground nut oil), safflower oil, sesame oil, soybean oil, sunflower oil, almond oil, cashew oil, hazelnut oil, macadamia oil, pecan oil, pistachio oil, walnut oil, acai oil, blackcurrant seed oil, borage seed oil, evening primrose oil, carob seed pods, amaranth oil, apricot oil, argan oil, avocado oil, babassu oil, ben oil, carob pod oil (algaroba oil), coriander seed oil, false flax oil (made of the seeds of camelina sativa), coriander seed oil, hemp oil, kapok seed oil, meadowfoam seed oil, mustard oil (pressed), okra seed oil, perilla seed oil, pine seed oil, poppyseed oil, prune kernel oil, pumpkin seed oil, quinoa oil, ramtil oil, rice bran oil, tea oil (camellia oil), thistle oil, wheat germ oil, castor oil, radish oil, ramtil oil, allanblackia oil, tung oil, shea butter, cocoa butter and combinations thereof.

Preferably, the vegetable oil is chosen from the group of oils consisting of soybean oil, sunflower oil, coconut oil, rapeseed oil, almond oil, babassu oil and combinations thereof.

Excellent results may be obtained when the vegetable oil is a high oleic oil, i.e. an oil containing an amount of linoleic acid (C18:2) of at least 4 wt %, more preferably at least 8 wt %, most preferably at least 12 wt % relative to the total weight of said oil. Preferably, said oil contains at least 25 wt % oleic acid, more preferably at least 50 wt % oleic acid, most preferably at least 75 wt % oleic acid relative to the total weight of said oil. Preferably, the vegetable oil contains at least at least 4 wt % linoleic acid, more preferably at least 8 wt % linoleic acid, most preferably at least 12 wt % linoleic acid and at least 25 wt % oleic acid. Non-limiting example of sources for the high oleic vegetable oil include sunflower, canola, peanut, safflower, corn, soybean, grapeseed, olive. Preferred high oleic oils include those produced from rapeseed, sunflower, soybean and combinations thereof.

The vegetable oil used in accordance with the invention can be modified or non-modified. Examples of modified oils include hydrogenated oils, fractionated oils and trans-esterified oils. In case a modified oil is used, the preferred modification is hydrogenation.

The inventive formulation preferably contains a texturizer. By “texturizer” is herein understood a component which has a predominant role in defining the texture of a product comprising thereof. Under certain circumstances, the texturizer may also stabilize the inventive formulation, in particular reduce “creaming” thereof, e.g. accumulation of the oil droplets at the surface of the formulation. It is however, important that the oil droplets do not coalesce, in other words the emulsifier composition is able to effectively stabilize said droplets. With the “texture of a product” is herein understood the general structure and disposition of the constituent parts of said product and refers to those qualities of the product that can be felt with the fingers, skin, tongue, palate or teeth.

The amount of texturizer is typically chosen to provide a desired texture to the inventive formulation and therefore it can vary within wide ranges to cover liquid textures but also semi-solid and solid textures. The texturizer is preferably present in the inventive formulation in an amount of at least 0.01 wt %, more preferably at least 0.05 wt %, even more preferably at least 0.07 wt %, most preferably at least 0.10 wt % relative to the total weight of said formulation. Preferably, the amount of texturizer is at most 90.00 wt %, more preferably at most 70.00 wt %, even more preferably at most 50.00 wt %, even more preferably at most 30.00 wt %, most preferably at most 10.00 wt %. Preferably the amount of texturizer is between 0.01 and 90.00 wt %, more preferably between 0.05 and 50.00 wt %, most preferably between 0.10 and 10.00 wt %.

Non-limiting examples of texturizers include starches, hydrocolloids, proteins, and like. Preferred texturizers are plant or algae derived, most preferred texturizer being a polysaccharide. Non-limiting examples of polysaccharides include xanthan gum (obtained by fermentation of Xanthomonas campestris); sclerotium gum (obtained by fermentation of Sclerotium rolfsii on a glucose substrate); pullulan (obtained by fermentation of Aurebasidium pullulans); tragacanth gum (which may be obtained from the dried sap of several species of Middle Eastern legumes of the genus Astragalus, including A. adscendens, A. gummifer, A. brachycalyx and A. tragacanthus); carrageenan gum (Carrageenan being a cell wall hydrocolloid found in several species of seaweeds belonging to red algae of the class: Rhodophyceae); alginates (which may be extracted from seaweed/Algea); konjac gum (which may be obtained from konjac tree of the genus Amorphophallus); agar-agar (found in cell walls of agarophyte red algae); gum Arabic (which may be obtained of hardened saptaken from two species of the acacia tree: Acacia Senegal and Acacia seyal); guar gum (a primary component of the ground endosperm of guar beans); starch; gellan gum (produced by Sphingomonas elodea); pectin (mainly from citrus and apple extract); cellulose (from a variety of plants, tree pulp and cotton linters); welan and dituan gum (obtained by aerobic fermentation); locust bean gum extract seeds of the carob tree; dammar gum (obtained from the Dipterocarpaceae family of trees in India and East Asia, principally those of the genera Shorea, Balanocarpus or Hopea); kauri gum (a fossilized resin detracted from kauri trees); spruce gum (obtained from a the resin of spruce trees); gum from Fenugreek; and gum anima (western or eastern).

Preferably, the texturizer contains xanthan gum. More preferably, the texturizer contains xanthan gum and a second polysaccharide, wherein the second polysaccharide is different than xanthan gum. Preferably, said second polysaccharide is chosen from the group consisting of a polyglucomannan polysaccharide such as sclerotium gum, pullulan and Konjac polyglucomannan; starch; alginate; carrageenan gum; pectin; gellan gum and gum Arabic.

More preferably, the texturizer contains sclerotium gum. Even more preferably, the texturizer contains sclerotium gum and a second polysaccharide, wherein the second polysaccharide is different than sclerotium gum. Preferably, said second polysaccharide is chosen from the group consisting of a xanthan gum, pullulan gum and Konjac gum; starch; alginate; carrageenan gum; pectin; gellan gum and gum Arabic. Most preferably, the texturizer contains xanthan gum and sclerotium gum.

It was observed that the preferred texturizers, may synergistically interact with the other ingredients of the inventive formulation to enhance the properties and characteristics thereof.

In one embodiment, the inventive formulation further contains water. The water is preferably present in an amount of at least 15 wt %, more preferably at least 30 wt %, even more preferably at least 40 wt %, most preferably at least 50 wt % relative to the total weight of said formulation. Preferably, the amount of water is at most 98 wt %, more preferably at most 96 wt %, even more preferably at most 94 wt %, even more preferably at most 92 wt %, most preferably at most 90 wt %. Preferably the amount of water is between 15 and 98 wt %, more preferably between 30 and 95 wt %, most preferably between 50 and 90 wt %.

In another embodiment, the inventive formulation is a dry formulation, i.e. it contains water in an amount of at most 15 wt %, more preferably at most 10 wt %, most preferably at most 8 wt % relative to the total weight of said formulation.

The inventive formulation may further contain a hydrating, humectant or moisturizing compound, non-limiting examples thereof including naturally occurring skin lipids and sterols, as well as artificial or natural oils; emollients; lubricants; polyols and polyol derivatives; hyaluronic acid and its salts; Chitosan; botanicals, e.g. Aloe Vera, Ginkgo, green tea extract, rose extract, sugar extract; polyethylene glycol ether of methyl glucose, e.g., Methyl Gluceth-10; green algae; natural and herbal extracts e.g. alpha lipoic acid, peptides, proteins amino acids and collagens and betaine; and combinations thereof.

Preferably, the inventive formulation contains a polyol or a polyol derivative as the hydrating, humectant or moisturizing compound. Preferably, said polyol or polyol derivative is chosen from the group of compounds consisting of glycerol, diglycerol, triglycerol, ethylene glycol, propylene glycol, butylene glycol, pentylene glycol (sometimes referred to herein as 1,2-pentane diol), isopreneglycol (1,4-pentane diol), 1,5-pentane diol, hexylene glycol, erythritol, 1,2,6-hexanetriol, polyethylene glycols such as PEG-4, PEG-6, PEG-7, PEG-8, PEG-9, PEG-10, PEG-12, PEG-14, PEG-16, PEG-18, PEG-20, combinations thereof, sugars and sugar derivatives (including fructose, glucose, maltose, maltitol, mannitol, inositol, sorbitol, sorbityl silanediol, sucrose, trehalose, xylose, xylitol, glucuronic acid and salts thereof), ethoxylated sorbitol (Sorbeth-6, Sorbeth-20, Sorbeth-30, Sorbeth-40), and combinations thereof.

Preferably the hydrating, humectant or moisturizing compound is a at least one naturally-obtained compound selected amongst sugars and other polyols, more preferably, said naturally-obtained compound is chosen from the group consisting of fructose, mannose, sucrose, glucose, dextrose, trehalose, mannitol, lactose, rhamnose (which may be obtained from a great verity of plant sources), sorbitol (which may be extracted, e.g., from seaweed and various fruits such as grapes, cherries, plums, apples, apricots, peaches, dates) and honey extracts, glycol, diols and polyols such as vegetable glycerin, 1,2-butanediol, propylene glycol, ethanol (which may be obtained from plant extracts or via fermentation), betaine, natural urea, lactic acid and other alpha hydroxy acids (such as glycolic acid, citric acid, mandelic acid, tartaric acid), colloidal oat meal, Aloe vera, hyaluronic acid, Panthenol, pyroglutamic acid (PCA) and its salts, and yeast and algae extract and combinations thereof.

Most preferred hydrating, humectant or moisturizing compound is glycerine (glycerol), in particular vegetable glycerine.

Preferably the hydrating, humectant or moisturizing compound is present in an amount of at least 1 wt %, more preferably at least 2 wt %, even more preferably at least 3 wt %, most preferably at least 5 wt % relative to the total weight of said formulation. Preferably, the amount of said compound is at most 40 wt %, more preferably at most 30 wt %, even more preferably at most 25 wt %, even more preferably at most 20 wt %, most preferably at most 15 wt %. Preferably the amount of said compound is between 1 and 40 wt %, more preferably between 3 and 20 wt %, most preferably between 5 and 15 wt %.

When used, preferably the glycerine is present in an amount of at least 1 wt %, more preferably at least 2 wt %, even more preferably at least 3 wt %, most preferably at least 5 wt % relative to the total weight of said formulation. Preferably, the amount of glycerine is at most 40 wt %, more preferably at most 30 wt %, even more preferably at most 25 wt %, even more preferably at most 20 wt %, most preferably at most 15 wt %. Preferably the amount of glycerine is between 1 and 40 wt %, more preferably between 3 and 20 wt %, most preferably between 5 and 15 wt %. Preferably, the glycerine is vegetable glycerine.

The inventive formulation may further contain an active ingredient, non-limiting examples thereof including cosmetically suitable anti-oxidants; vitamins; plant or botanical extracts; and other ingredients having an anti-aging effect; skin soothing; skin whitening, e.g. hydroquinone, kojic acid and arbutin; skin repair compounds; skin tone; anti-cellulite effect; anti-inflammatory effect; and the like. Specific examples thereof are given in WO 2017/027570 in paragraph [0035], the entire disclosure thereof being included herein by reference.

The inventive formulation may also comprise at least one preservative, non-limiting examples thereof including ethylparaben; imidazofidinyl urea; methylparaben, sorbic acid and its salts, O-phenylphenoi; propylparaben; quaterntum-14; quatemium-5; sodium dehydroacetate; pnenoxyethanoi, phenoxyisopropanol, benzyl alcohol, polyaminopropyl biguanide, triethylene glycol, piroctone olamine, benzoic acid and its salts, dehydroacetic acid and its salts, diazolidinyl urea, iodopropynyl butyicarbamate, rnethylisothfazolinone, glyceryl caprylate, phenylethylalcohol, caprylyl glycol, capryloyl glycine, phenylpropanol, ethylhexylglycerin, ethylpropanediol, and their salts and their blends.

The inventive formulation may also comprise perfumes; solvents, e.g. alcohols; sunfilter or sunscreen materials, e.g. those based on titanium dioxide or zinc oxide; skin care agents, e.g. ceramides; germanium containing compounds; caffeine; cooling materials, e.g. menthol or camphor; insect repellents; essential oils; and pigments as well as other compounds as disclosed in WO 2017/055943, the disclosure of which is herein incorporated by reference.

The inventive formulation is preferably in an emulsion form. The emulsion form can be formulated either as a simple emulsion, which may be texturized as is described above, or it can be formulated in more complex systems such as suspoemulsions or multiple emulsions. Suspoemulsions include a liquid dispersed phase and a solid dispersed phase. The solid phase can contain a pigment; or a physical sunscreen of a metal oxide such as titanium and/or aluminium and/or zinc oxides. The invention therefore relates to a personal care formulation in emulsion form, the emulsion comprising an emulsifier composition, a vegetable oil and optionally a texturizer, wherein the emulsifier composition contains an Acetone Insoluble (AI) component, said AI component containing at least Phosphatidyl Choline (PC) and Phosphatidyl Ethanolamine (PE), wherein the emulsifier composition is of a vegetable origin, wherein the emulsifier composition is characterized by a weight ratio R of at least 5.0, the ratio R being defined according to Formula 1:

$$R = \frac{{W}_{PC}}{{W}_{PE}} \qquad Formula 1$$

wherein W_{PC} and W_{PE} are the individual weights of the respective PC and PE relative to the total weight of the emulsifier composition. Preferably, the emulsifier composition utilized in the emulsion has an R of between 5 and 100, an AI between 30 and 90 wt % and a PC content of between 20 and 65 wt %. More preferably, said emulsifier composition has an R of between 5 and 100, an AI between 30 and 90 wt %, a PC content of between 20 and 65 wt %, a PE content of between 0.1 and 10 wt % and a PA content of between 0.1 and 3.0 wt %. Even more preferably, said emulsifier composition has an R of between 5 and 100, an AI between 30 and 90 wt %, a PC content of between 20 and 65 wt %, a PE content of between 0.1 and 10 wt %, a PA content of between 0.1 and 3.0 wt % and a PI content of between 0.1 and 10.0 wt %. Most preferably, said emulsifier composition has an R of between 5 and 100, an R′ of between 4.0 and 100.0, an AI between 30 and 90 wt %, a PC content of between 20 and 65 wt %, a PE content of between 0.1 and 10 wt %, a PA content of between 0.1 and 3.0 wt % and a PI content of between 0.1 and 10.0 wt %. Preferred ranges and combinations of ranges for AI, PC, PE, PA, PI, R and R′ are given above and will not be repeated herein.

Preferably, the emulsion is an oil-in-water (O/W) emulsion, said emulsion comprising a continuous aqueous phase and a discontinuous oil phase dispersed within said continuous aqueous phase. Accordingly, a “discontinuous oil phase” refers to the multiplicity of discrete elements dispersed within, and immiscible with, the continuous aqueous phase. The discontinuous oil phase is present in the form of particles. As used herein, a “particulate” refers to an oil phase of an emulsion that comprises a plurality of discrete particles. As used herein, the term “particle size” refers to particle diameter, which is the diameter of the particles based on an approximate spherical shape of the particle based on a volumetric measurement of the particle. In addition to spherical particles, the oil-in-water emulsions of the present invention can also comprise without limitation semi-spherical, ellipsoidal and/or cylindrical particles.

The stability of the emulsion is assessed by determining whether there has been a change in the particle size distribution of the emulsion over time but also particle aggregation and particle accumulation at interfaces. A lack of change, or a relatively small change, in particle size distribution of the emulsion over time, or a lack (or relatively small level) of accumulation and/or aggregation indicate that the emulsion is stable. The present inventors determined that the emulsions of the invention are stable over time.

As used herein, a particle size “distribution” refers to the number or concentration (e.g., percentage) of particles having a certain size (i.e., diameter), or range of sizes, within a given emulsion, lot and/or batch of the present invention. As used herein, a particle size “distribution” refers to the number or concentration (e.g., percentage) of particles having a certain size (i.e., diameter), or range of sizes, within a given emulsion, lot and/or batch of the present invention. Particle size and particle size distribution can be measured using Laser Light Scattering (LLS) with, for example, a Malvern Mastersizer 3000 hydro module, Malvern Instruments Ltd., Worcestershire, UK). Particle size and particle size distribution can also be measured by, for example, micro-photography, video microscopy, video-enhanced microscopy, Coulter counting, differential scanning calorimetry, turbidimetry, dynamic and/or static light scattering, low-intensity ultrasound, nuclear magnetic resonance, or any other particle size measurement technique known to persons of ordinary skill in the art.

As used herein, a “D50” or “Dv(0.5) value refers to the particle size of an oil phase, and specifically the diameter at which 50% of the measurable particles of the oil phase particles have a larger equivalent diameter, and the other 50% of the particles have a smaller equivalent diameter. The particles are considered spherical. Thus, D50 generally refers to the median particle diameter.

Preferably, the emulsion of the invention has a D50 that is at least 10% smaller than an equivalent formulation, which with the exception of the emulsifier composition, it contains exactly the same ingredients in exactly the same quantities and is prepared in exactly the same way, more preferably at least 20%, even more preferably at least 30%, most preferably at least 40%. The inventors observed that the emulsion of the invention can be produced to contain particles sizes of the oil phase that are smaller and better dispersed than said equivalent formulations.

Preferably, the emulsion of the invention is a 0/W emulsion having a D50 of at least 0.1 um, more preferably at least 0.5 um, even more preferably at least 1.0 um, yet yen more preferably at least 1.5 um, most preferably at least 2.0 um. Preferably, D50 is at most 5.0 um, even more preferably at most 4.0 um, most preferably at most 3.0 um.

The particle distribution width or breadth is calculated as a SPAN value, in accordance with Formula 1:

$$SPAN = \frac{D 90 - D 10}{D 50} \qquad Formula 1$$

As used herein, a “D90” or “Dv(0.9) value refers to the particle size of an oil phase, and specifically the diameter at which 90% of the measurable particles of the oil phase particles have a smaller equivalent diameter.

As used herein, a “D10” or “Dv(0.1) value refers to the particle size of an oil phase, and specifically the diameter at which 10% of the measurable particles of the oil phase particles have a smaller equivalent diameter.

Preferably, the emulsion of the invention has a SPAN of at least 10% smaller than an equivalent formulation, which with the exception of the emulsifier composition, it contains exactly the same ingredients in exactly the same quantities and is prepared in exactly the same way, more preferably at least 20%, even more preferably at least 30%, most preferably at least 40%.

Preferably, the emulsion of the invention is a 0/W emulsion having a D50 of between 0.1 and 5.0 um and a SPAN of at most 4.5, more preferably at most 4.0, most preferably at most 3.7. Preferably, said D50 is at least 0.5 μm, even more preferably at least 1.0 μm, yet yen more preferably at least 1.5 μm, most preferably at least 2.0 μm. Preferably, D50 is at most 4.0 μm, most preferably at most 3.0 μm.

Preferably, the inventive formulation is a cream and contains between 0.5 and 8 wt % of the emulsifier composition, between 10 and 30 wt % of the vegetable oil and between 1 and 3 wt % of the texturizer, wherein the R ratio of the emulsifier composition is between 5 and 100. Preferred values for the R values are given above and will not be repeated herein.

Preferably, the inventive formulation is a serum and contains between 2 and 10 wt % of the emulsifier composition, between 1 and 5 wt % of the vegetable oil and between 0.3 and 1 wt % of the texturizer, wherein the R ratio of the emulsifier composition is between 5 and 100.

Preferably, the inventive formulation is a body milk and contains between 2 and 8 wt % of the emulsifier composition, between 5 and 20 wt % of the vegetable oil and between 0.5 and 2 wt % of the texturizer, wherein the R of the emulsifier composition is between 5 and 100.

Preferably, the inventive formulation is a lip balm and contains between 0.2 and 8 wt % of the emulsifier composition, between 50 and 85 wt % of the vegetable oil and between 15 and 50 wt % of the hydrogenated vegetable oil, wherein the R ratio of the emulsifier composition is between 5 and 100.

The inventive formulations may be used in a variety of conventional manners, e.g. for cleansing and conditioning the hair and/or skin, and other keratinous material such as nails, but also lips. The formulations may be applied to the desired area of the skin, hair or nails in an amount sufficient to provide the desired effect. The application of the formulation may be directly onto the skin, hair or nails, or indirectly via the use of an implement such as a cosmetic puff, a washcloth, a patch, a sticker, a wipe or a sponge.

The invention further relates to a method of manufacturing the inventive formulation comprising the steps of:
a. Providing an aqueous phase containing the texturizer and water
b. Providing an oil phase containing the vegetable oil;
c. Adding the emulsifier composition to the water phase and/or to the oil phase, preferably said emulsifier composition is added to the oil phase; and then
d. Mixing the oil phase with the aqueous phase

At step a. of the method of the invention (hereinafter the “inventive method”) the texturizer is preferably homogeneously dispersed in the water, preferably with the help of a mixer. Preferably, the texturizer and/or the water are heated up before mixing to a temperature of between 30 and 95° C., more preferably between 50 and 90° C., most preferably between 70 and 85° C. The dispersion, preferably the heated dispersion, is preferably subjected to a high shear treatment, e.g. by using a mixer such as Ultra-Turrax Mixer at between 2.000 and 20.000 rpm, more preferably between 7.000 and 16.000 rpm.

At step b. of the inventive method the emulsifier composition is preferably dissolved or dispersed in the vegetable oil at a temperature of preferably between 30 and 95° C., more preferably between 50 and 90° C., most preferably between 70 and 85° C.

At step c. of the inventive method, the oil phase is mixed with the aqueous phase to preferably form an 01W emulsion, i.e. to disperse the oil inside the water phase to form oil particulates in a continuous water phase. Preferably, the oil phase is mixed with the aqueous phase under high shear, e.g. by using a mixer such as Ultra-Turrax Mixer at between 2.000 and 20.000 rpm, more preferably between 7.000 and 16.000 rpm. Preferably, the mixing is carried out at a temperature of between 30 and 95° C., more preferably between 50 and 90° C., most preferably between 70 and 85° C.

After cooling down the inventive formulation to room temperature, following step c. of the inventive method, further ingredients as listed hereinabove can be added thereto.

The wt % of the various ingredients depends on the intended use of the inventive formulation and is indicated hereinabove. For example, in order to obtain a cream-like texture, the texturizer is preferably added to the inventive formulation in an amount of between 1 and 3 wt %, while the vegetable oil is preferably used in an amount of between 10 and 30 wt %. For a serum-like texture, the texturizer is preferably added to the inventive formulation in an amount of between 0.3 and 1 wt %, while the vegetable oil is preferably used in an amount of between 1 and 5 wt %. For a body milk-like texture, the texturizer is preferably added to the inventive formulation in an amount of between 0.5 and 2 wt %, while the vegetable oil is preferably used in an amount of between 5 and 20 wt %.

The inventors surprisingly observed that by combining the texturizer, vegetable oil and emulsifier composition in the quantities mentioned above, the texture of the inventive formulation can be varied form liquid-like to solid-like. Such a versatility was to inventors' knowledge hardly achieved by other personal care formulations hitherto.

The invention further relates to personal care products containing the inventive formulation. Personal care products include skin creams, soaps, soap bars, bath and shower gels, shampoos, mousses, deodorants, anti-perspirants, lipsticks, sunscreens and oral care products such as toothpastes and mouthwashes but also those mentioned hereinabove.

The invention will be further explained with the help of the following Examples and Comparative Experiments, without however being limited thereto.

### Methods for Measurement

D50, D90, D10 and SPAN are measured using Laser Light Scattering (LLS) with a Malvern Mastersizer 3000 hydro module, from Malvern Instruments Ltd., Worcestershire, UK.
Acetone insolubles were determined according to Lange R., Fiebig H. J. (1999): Separation of Phospholipids, Standard Methods of DGF, Fett/Lipid 101: 77-79. This method is based on the solubility of lecithin components such as triglycerides, fatty acids, sterols, and other acetone-soluble components, and the insolubility of the phospholipids and glycophospholipids in acetone under the test conditions. The latter are termed acetone insolubles (AI). AI may also be determined in accordance with AACC International Method 58-35.01—“Acetone-Insoluble Lecithin”, however the former method is preferred.
Phospholipid Composition: The phospholipid composition, i.e. the amount of PC, PA, PI and PE and their hydrolysed fractions was determined using a liquid-chromatographic method applied on emulsifier compositions having an AI set to 60% relative to the total weight of the emulsifier composition. AI amount can be adjusted by adding (or extracting e.g. with acetone) the necessary amount of the acetone soluble part (mainly triglycerides) of said composition in order to bring the AI amount to 60%. The identification and quantification of the various phospholipid components may conveniently be executed by different methods, including thin-layer chromatography (TLC), high performance liquid chromatography (HPLC) and ³¹P nuclear magnetic resonance spectroscopy (³¹P-NMR) for the phospholipids only. Suitable methods are disclosed in London E., Feigenson G. W. (1979): Phosphorous NMR Analysis of Phospholipids in Detergents, J. Lipid Res. 20: 408-412; Aitzetmüller K. (1984): HPLC and Phospholipids, Part I: General Considerations, Fette, Seifen, Anstrichm. 86: 318-322; and Aloisi J. D., Sherma J., Fried B. (1990): Comparison of Mobile Phases for Separation and Quantification of Lipids by One-Dimensional TLC and Preadsorbent High Performance Silica Gel Plates, J. Liq. Chromatogr. 13:3949-3961.
Stability of a formulation is_measured after keeping said formulation for 2 months at 45° C. The ranking is established as follows:
   lower particle size (measured as D50 with Malvern Mastersizer 3000 hydro module), a ranking is established following the below criteria:
      9=D50 below 3.5 μm
      1=D50 above 3.5 μm
   narrower particle size distribution (meaning smaller SPAN measured with Malvern Mastersizer 3000 hydro module) a ranking is established following the below criteria:
      9=SPAN<2
      6=2≤SPAN<3
      3=3≤SPAN<4
      1=SPAN≥4
   visually stable: visual observations recorded and ranked by the technician as follows after 2 months of exposure at 45 C:
      9=no oil droplets or coalescence, no creaming
      6=no oil droplets or coalescence, creaming
      3=oil droplets or coalescence

3 1=continuous oil phase or oil layer

### EXAMPLES AND COMPARATIVE EXPERIMENTS

### Example 1

### Production of a Cream

TABLE 1

| | COMPOSITION |
|---|---|
| INCI NAME | (wt %) |
| Aqua (demi) | 79.14 |
| Glycerine | 8.00 |
| Texturizer 1 (Xanthan + Sclerotium gum) | 1.00 |
| Brassica Campestris Seed Oil | 10.00 |
| Emulsifier composition 1 (EC1) | 1.00 |
| Optional Phenoxyethanol (and) | 0.50 |
| Ethylhexylglycerin | |
| Optional Fragrance (Diamond 256327) | 0.18 |
| Optional Pigment (Puricolor Red ARE33) | 0.18 |
| TOTAL | 100 |

The aqueous phase consisting of water, glycerine and texturizer was formed by mixing the components under agitation. The mixture was heated up to 70° C. and subjected for about 10 minutes to high shear treatment in an Ultra-Turrax operating at 19.000 rpm. The oil phase consisting of the oil and EC1 was formed by heating up to 70 C and mixing the components for 20 min.

The cream was produced by mixing for about 10 minutes, the oil phase and the water phase at a temperature of about 70° C. in an Ultra-Turrax operating at 18.000 rpm. After cooling to room temperature, the Phenoxyethanol (and) Ethylhexylglycerin, fragrance and the colour were added under gentle mixing at 1000 rpm with 4 bladed helix.

The EC1 had a composition as detailed in Table 5.

### Example 2

### Production of a Serum

TABLE 2

| | COMPOSITION |
|---|---|
| INCI NAME | (wt %) |
| Aqua (demi) | 87.01 |
| Glycerine | 8.00 |
| Texturizer 1 (Xanthan + Sclerotium gum) | 0.30 |
| Glycerine | 8.00 |
| Brassica Campestris Seed Oil | 2.00 |
| Emulsifier composition 1 (EC1) | 2.00 |
| Optional Phenoxyethanol (and) | 0.50 |
| Ethylhexylglycerin | |
| Optional Fragrance (Puricolor Blue ABL9-X) | 0.13 |
| Optional Plant extract (Green Tea 774925) | 0.06 |
| TOTAL | 100 |

The aqueous phase consisting of water, glycerine and texturizer was formed by mixing the components under agitation. The mixture was kept at room temperature and subjected for about 10 minutes to high shear treatment in an Ultra-Turrax operating at 18.000 rpm. The oil phase consisting of the oil and EC1 was formed by mixing the components for 20 min at room temperature.

The serum was produced by mixing for about 10 minutes, the oil phase and the water phase at room temperature in an Ultra-Turrax operating at 18.000 rpm. The Phenoxyethanol (and) Ethylhexylglycerin, fragrance and the colour were added in a last step under gentle mixing at 1000 rpm with 4 bladed helix.

### Example 3

### Production of a Body Milk

TABLE 3

| | COMPOSITION |
|---|---|
| INCI NAME | (wt %) |
| Aqua (demi) | 78.80 |
| Glycerine | 8.00 |
| Texturizer 1 (Xanthan + Sclerotium gum) | 0.50 |
| Brassica Campestris Seed Oil | 10.00 |
| Emulsifier composition 1, 6, 7 and 8 | 2.00 |
| Optional Phenoxyethanol (and) | 0.50 |
| Ethylhexylglycerin | |
| Optional Plant extract (Cucumber Green Tea 316686) | 0.20 |
| TOTAL | 100 |

The aqueous phase consisting of water, glycerine and texturizer was formed by mixing the components under agitation. The mixture was heated up to 70° C. and subjected for about 10 minutes to high shear treatment in an Ultra-Turrax operating at 19.000 rpm. The oil phase consisting of the oil and EC was formed by heating up to 70 C and mixing the components for 20 min.

The body milk was produced by mixing for about 10 minutes, the oil phase and the water phase at a temperature of about 70° C. in an Ultra-Turrax operating at 18.000 rpm. After cooling to room temperature, the Phenoxyethanol (and) Ethylhexylglycerin, fragrance and the colour were added under gentle mixing at 1000 rpm with 4 bladed helix.

### Example 4 and Comparative Examples (CE)

TABLE 4

| | Ex 4 | Ex 1 | Ex 2 | Ex 3 | CE 1 | CE 2 | CE 3 | CE 4 |
|---|---|---|---|---|---|---|---|---|
| INCI NAME | wt % | | | | | | | |
| Aqua (demi) | 77.50 | | | | 77.50 | 77.50 | 77.50 | 77.50 |
| Glycerine | 8.00 | | | | 8.00 | 8.00 | 8.00 | 8.00 |
| Sweet Almond oil | 10.00 | | | | 10.00 | 10.00 | 10.00 | 10.00 |
| EC 1 | 4.00 | — | | | — | — | — | — |
| EC 2 | | | | | 4.00 | — | — | — |
| EC 3 | | | | | — | 4.00 | — | — |
| EC 4 | | | | | — | — | 4.00 | 0 |
| EC 5 | | | | | — | — | — | 4.00 |
| EC 6 | | 4.00 | | | — | | | |
| EC 7 | | | 4.00 | | | | | |
| EC 8 | | | | 4.00 | | | | |
| Optional | 0.50 | | | | | | | |
| Phenoxyethanol | | | | | | | | |
| (and) Methylparaben | | | | | | | | |
| (and) Ethylparaben | | | | | | | | |
| (and) Butylparaben | | | | | | | | |
| (and) Propylparaben | | | | | | | | |
| Optional Sodium | q.s.p. | q.s.p. | q.s.p. | q.s.p. | q.s.p. | q.s.p. | q.s.p. | q.s.p. |
| hydroxyde | pH 5.5 | pH 5.5 | pH 5.5 | pH 5.5 | pH 5.5 | pH 5.5 | pH 5.5 | pH 5.5 |
| TOTAL | 100 | 100 | 100 | 100 | 100 | 100 | 100 | 100 |

### Stability Results

A comparison was made to study the stability of Body milk formulations using various emulsifier compositions as presented in Table 5. All emulsifying composition were of vegetable origin. Most preferred compositions are those derived from soy, sunflower and rapeseed. The ECs used in the examples have a lipid component in an amount of (100%—AI) and having the same origin as the phospholipid composition of the EC.

The RESULTS are the sum of ranking, the higher the ranking the better the stability. The utilization of emulsifier compositions in accordance with the invention provided the best stability. In all inventive experiments the oil droplets were not coalescing and their sizes (as measured by D50 and SPAN) remained substantially constant during the testing period.

The stability was investigated by measuring D50, span and visually inspecting the sample.

The inventors observed that by using an EC according to the invention, the stability was enhanced. The results were confirmed by visual observations carried out after accelerated stability evaluation by exposing the sample for 2 months at 45° C.

TABLE 5

| | | | | | | | | | | D50 @ 2 | SPAN | | |
|---|---|---|---|---|---|---|---|---|---|---|---|---|---|
| | Al | PC | PE | PA | PI | LPC | | | | months | @ 2 | Visual | RESULTS |
| | wt % | | | | | | R | R’ | | μm | months | observation | (ranking) |
| EC1 | 50.3 | 32.2 | 3.4 | 0.5 | 0.6 | 2.0 | 9.47 | 7.15 | Ex 4 | 2.8 | 3.7 | creaming | 18 |
| EC2 | 63.83 | 16.59 | 13.48 | 2.34 | 8.38 | 0.76 | 1.23 | 0.68 | CE 1 | 5.39 | 3.39 | creaming, | 13 |
| | | | | | | | | | | | | few oil | |
| | | | | | | | | | | | | droplets on | |
| | | | | | | | | | | | | top | |
| EC3 | 60.5 | 14.1 | 8.4 | 3.1 | 11.2 | 1.1 | 1.67 | 0.62 | CE 2 | 3.34 | 4.75 | creaming, oil | 13 |
| | | | | | | | | | | | | aggregating | |
| | | | | | | | | | | | | on top | |
| EC4 | 94.7 | 92.5 | <0.1 | <0.1 | <0.1 | 0.6 | 925 | 308 | CE 3 | 12.17 | 6.29 | continuous | 7 |
| | | | | | | | | | | | | oil phase or | |
| | | | | | | | | | | | | oil layer | |
| EC5 | 96.4 | 96.2 | <0.1 | <0.1 | <0.1 | 0.3 | 962 | 320 | CE 4 | 14.45 | 1.93 | continuous | 3 |
| | | | | | | | | | | | | oil phase or | |
| | | | | | | | | | | | | oil layer | |
| EC6 | 51.1 | 29.8 | 3.8 | 0.8 | 1.3 | 2.2 | 7.8 | 5.1 | Ex 1 | 2.7 | 2.8 | creaming | 20 |
| EC7 | 59.5 | 45.0 | 2.7 | 0.4 | 0.5 | 1.6 | 16.6 | 12.5 | Ex 2 | 2.2 | 2.9 | creaming | 25 |
| EC8 | 76.0 | 61.1 | 3.7 | 0.6 | 1.8 | 0.9 | 16.5 | 9.9 | Ex 3 | 2.7 | 4.3 | creaming | 19 |

1. A personal care formulation comprising an emulsifier composition, a vegetable oil and optionally a texturizer, wherein the emulsifier composition contains an Acetone Insoluble (AI) component, said AI component containing at least Phosphatidyl Choline (PC) and Phosphatidyl Ethanolamine (PE), wherein the emulsifier composition is of vegetable origin, wherein the emulsifier composition is characterized by a weight ratio R of at least 5.0, the ratio R being defined according to Formula 1:
$$R = \frac{{W}_{PC}}{{W}_{PE}} \qquad Formula 1$$
wherein W_{PC} and W_{PE} are the individual weights of the respective PC and PE relative to the total weight of the emulsifier composition.

2. The formulation of claim 1 wherein the AI component is in a weight ratio of at least 30 wt % relative to the total weight of the emulsifier composition.

3. The formulation of claim 1 wherein the PC is in an amount of at least 20 wt % relative to the total weight of said emulsifier composition.

4. The formulation of claim 3 wherein the PE is in an amount of at most 10.0 wt %.

5. The formulation of claim 3 wherein said formulation also contains a Phosphatidic Acid (PA) and wherein the PA is in an amount of at most 3.0 wt %.

6. The formulation of claim 1 wherein said formulation also contains a Phosphatidyl Inositol (PI) and wherein the PI is in an amount of at most 10.0 wt %.

7. The formulation of claim 1 containing a texturizer, wherein the texturizer contains xanthan gum and a second polysaccharide, wherein the second polysaccharide is different than xanthan gum.

8. The formulation of claim 7 wherein the texturizer is in an amount of at least 0.01 wt %.

9. The formulation of claim 3 wherein the vegetable oil is chosen from the group consisting of coconut oil, corn oil, cottonseed oil, canola oil (rapeseed oil), olive oil, palm oil, peanut oil (ground nut oil), safflower oil, sesame oil, soybean oil, sunflower oil, almond oil, cashew oil, hazelnut oil, macadamia oil, pecan oil, pistachio oil, walnut oil, acai oil, blackcurrant seed oil, borage seed oil, evening primrose oil, carob seed pods, amaranth oil, apricot oil, argan oil, avocado oil, babassu oil, ben oil, carob pod oil (algaroba oil), coriander seed oil, false flax oil (made of the seeds of camelina sativa), coriander seed oil, hemp oil, kapok seed oil, meadowfoam seed oil, mustard oil (pressed), okra seed oil, perilla seed oil, pine seed oil, poppyseed oil, prune kernel oil, pumpkin seed oil, quinoa oil, ramtil oil, rice bran oil, tea oil (camellia oil), thistle oil, wheat germ oil, castor oil, radish oil, ramtil oil, allanblackia oil, tung oil, shea butter, cocoa butter and combinations thereof.

10. The formulation of claim 9 wherein the vegetable oil is present in an amount of at least 0.1 wt %.

11. The formulation of claim 10, said product further containing glycerin.

12. The formulation of claim 10, said product further containing a perfume, an active substance, a preservative, a colorant or a mixture thereof.

13. A personal care or cosmetic product containing the formulation of claim 10, said product having a texture of a cream, a texture of a body milk, a texture of a lip balm, a texture of a serum or a texture of a shampoo.

14. A cream, a body milk, a lip balm, a serum or a shampoo, containing the formulation of claim 10.
